(19) United States
(12) Patent Application Publication
(10) Pub. No.: US 2023/0406891 A1
(43) Pub. Date: Dec. 21, 2023

(54) Dual-functional Bean-derived Polypeptide and Preparation Method Therefor

(71) Applicant: Jiangnan University, Wuxi (CN)

(72) Inventors: Yuanfa Liu, Wuxi (CN); Zhaojun Zheng, Wuxi (CN)

(21) Appl. No.: 18/239,386

(22) Filed: Aug. 29, 2023

Related U.S. Application Data
(63) Continuation of application No. PCT/CN2022/087093, filed on Apr. 15, 2022.

(30) Foreign Application Priority Data
May 21, 2021 (CN) 2021105559680

(51) Int. Cl. C07K 14/415; A23J 1/14; A23J 3/14; A23J 3/34; A23D 7/005; A23D 7/04; A23L 33/18; C07K 1/14; A61K 38/16; A61K 8/64; C12P 21/06

(52) U.S. Cl. CPC C07K 14/415; A23J 1/14; A23J 3/14; A23J 3/346; A23D 7/0053; A23D 7/04; A23L 33/18; C07K 1/145; A61K 38/168; A61K 8/645; C12P 21/06

(57) ABSTRACT

The present disclosure discloses the dual-functional bean-derived polypeptides and a preparation method therefor, and belongs to the technical field of functional polypeptides. The method includes extracting a bean source protein isolate from beans by an alkali-solution and acid-isolation method first, then subjecting the bean source protein isolate to infrared heat treatment, and finally performing enzymolysis with a protease to obtain the bean-derived polypeptides. The bean-derived polypeptides have dual functions of an antihypertensive activity and an antioxidant activity at the same time.

### TECHNICAL FIELD

The present disclosure relates to the dual-functional bean-derived polypeptides and preparation method therefor, and belongs to the technical field of functional polypeptides.

### BACKGROUND

With people's pursuit of nutrition, health and sustainable development, plant-based foods are increasingly favored by people. The plant-based foods are foods prepared by using plant products as raw materials. As indispensable raw materials of the plant-based foods, plant proteins and derivatives thereof are also in continuously increasing demands. Beans are main sources of the plant proteins, and protein contents of the beans are about 20-40%, which are 1-3 times higher than cereals and 3-5 times higher than tubers. However, as natural bean proteins have complex and dense structures, application thereof in the food industry is greatly limited.

Therefore, seeking suitable methods for modifying the bean proteins is of great significance to improve nutritional and functional properties of the bean proteins.

Due to safety and effectiveness, enzymatic modification has been widely used in modification of food proteins to improve functional properties or physiological activities of the proteins. Enzymatic hydrolysis reactions are high in speed, high in safety, convenient to control and not able to weaken nutritional values of proteins, and can improve functional properties of the proteins to a certain extent. In addition, polypeptide substances produced by hydrolysis of proteins usually contain abundant small-molecule bioactive substances, which have biological activities of lowering blood pressure, resisting oxidation, resisting bacteria, realizing immune regulation and the like. These polypeptide substances having biological activities not only provide nutrient elements for the body, but also have important benefits for maintaining body health.

Consumption of the polypeptide substances having the effect of lowering blood pressure is conducive to preventing and assisting in the control of hypertension, so as to effectively prevent and curb the occurrence of cardiovascular diseases. However, at present, research of the polypeptide substances is mainly focused on mining of single activity or drug application, while research of dual-functional peptides and application research thereof in the food industry are relatively little.

### SUMMARY

The present disclosure discloses a method for preparing bean-derived source polypeptides having antihypertensive activity and antioxidant activity at the same time. The bean-derived polypeptides are the polypeptides prepared by using bean foods as the raw materials.

The present disclosure discloses a method for preparing the dual-functional bean-derived polypeptides. The method includes the following steps:

(1) extracting a bean source protein isolate from beans by an alkali-solution and acid-isolation method;

(2) subjecting the bean source protein isolate to infrared heat treatment at an infrared temperature of 70-140° C. for 10-60 min; and

(3) after the infrared heat treatment, subjecting the bean source protein isolate to enzymolysis with a protease to obtain the bean-derived polypeptides having dual functions of an antihypertensive activity and an antioxidant activity at the same time.

In one embodiment of the present disclosure, the method includes the following steps:

(1) extraction of a bean source protein isolate: subjecting a bean raw material to pulverizing and sifting, adding n-hexane for degreasing to obtain a degreased bean powder, then extracting a protein from the degreased bean powder by an alkali-solution and acid-isolation method, and performing freeze-drying to obtain a bean source protein isolate;

(2) infrared treatment: subjecting the bean source protein obtained in step (1) to infrared heat treatment for destroying an intermolecular force so as to denature a protein structure thereof; and

(3) preparation of a polypeptide: dissolving an infrared protein powder obtained in step (2) in deionized water to prepare a solution having a substrate concentration of 5-20%, adjusting the pH value, adding a plant protease to carry out an enzymolysis reaction, and after the reaction is completed, performing enzyme deactivation at a high temperature, followed by centrifugation to collect a polypeptide solution.

In one embodiment of the present disclosure, the bean raw material includes one or more of protein-rich soybeans, black beans, peas, red beans, or mung beans.

In one embodiment of the present disclosure, in step (1), a ratio of the material to the n-hexane during the degreasing is 1:3 to 1:10 (w/v, g/mL).

In one embodiment of the present disclosure, in step (1), the alkali-solution and acid-isolation method specifically includes: dissolving the degreased bean powder in deionized water to obtain a solution, adjusting the pH value of the solution to 7.2-10.5 with sodium hydroxide, stirring at a constant temperature of 25-50° C. for 0.5-4.5 h, followed by centrifugation to obtain a supernatant, then adjusting the pH value to 2.0-5.5 with hydrochloric acid, repeating the above steps for 2-3 times to obtain a precipitate, and washing the precipitate to neutral with deionized water.

In one embodiment of the present disclosure, in step (2), the infrared treatment may include performing flexible thermal processing on the protein to destroy an intermolecular force of the protein so as to stretch the protein, so that exposure of a hydrophobic group is facilitated, enzymolysis efficiency is improved, and convenience is provided for releasing bioactive substances. The infrared heat treatment is performed at an infrared temperature of for 10-60 min.

In one embodiment of the present disclosure, in step (3), the plant protease may include one or more of ficin, bromelain and papain, the protease is added in an amount of 500-5,000 U/g protein powder, and the reaction is preferably carried out at a temperature of 35-65° C. and a pH value of 4.5-7.5 for 0.5-8 h.

In one embodiment of the present disclosure, in step (3), the polypeptide solution is required to be refrigerated at a low temperature; and when not used in time, the polypeptide solution is required to be frozen at −80° C. to −10° C. for storage.

The present disclosure further discloses the dual-functional bean-derived polypeptides prepared by the above method. The bean-derived polypeptides have dual functions of an antihypertensive activity and an antioxidant activity at the same time.

The present disclosure further discloses a nano-emulsion containing bean-derived polypeptides. The emulsion is obtained by homogenization and mixing of the dual-functional bean-derived polypeptides and an oil phase. The-emulsion containing bean-derived polypeptides is a novel dual-functional plant-based product.

In one embodiment of the present disclosure, the oil phase includes an edible animal or plant oil, an essential oil and other fat-soluble nutrients.

In one embodiment of the present disclosure, a method for preparing-an emulsion containing bean-derived polypeptides includes the following steps:

(1) pre-preparation of a crude polypeptide emulsion: adjusting the above polypeptide solution to an appropriate concentration to obtain an aqueous phase solution, dissolving a rosemary extract in a vegetable oil to obtain an oil phase, mixing the aqueous phase with the oil phase, and performing high-speed shear dispersion at a rotation speed of 8,000-25,000 rpm for min to obtain a crude emulsion; and

(2) preparation of a polypeptide emulsion: subjecting the crude emulsion obtained in step (1) to pretreatment immediately by a high-pressure homogenizer, followed by ultrahigh-pressure micro-fluidization treatment to obtain a polypeptide nanoemulsion.

In one embodiment of the present disclosure, in step (1), the polypeptide solution has a protein concentration of 0.5-100 mg/mL, and the rosemary extract is added in an amount of g/kg.

In one embodiment of the present disclosure, in step (1), the vegetable oil may be a rapeseed oil, a corn oil, a flaxseed oil, a walnut oil, an olive oil, a sunflower seed oil, a seaweed oil, a peony seed oil, a perilla oil, a safflower oil, or a camellia oil.

In one embodiment of the present disclosure, in step (1), a mass ratio of the aqueous phase to the vegetable oil is 1:1 to 20:1 (v/v).

In one embodiment of the present disclosure, in step (2), the high-pressure homogenizer is set in a pressure range of (40-150)/(5-50) bar, and the emulsion is rapidly cooled to 2-10° C. in an ice water bath after homogenization.

In one embodiment of the present disclosure, in step (2), the high-pressure micro-fluidization treatment is performed at a pressure of 300-1,500 bar and circulated for 2-6 times.

The present disclosure further discloses a composition containing the dual-functional bean-derived polypeptides or the emulsion containing bean-derived polypeptides.

The present disclosure further discloses application of the dual-functional bean-derived polypeptides or the emulsion containing bean-derived polypeptides in preparation for foods and cosmetics.

The present disclosure further discloses a dual-functional bean-derived polypeptides microcapsule powder. The bean-derived polypeptides microcapsule powder is obtained by performing spray-drying treatment on the emulsion containing bean-derived polypeptides.

According to the method for preparing the dual-functional bean-derived polypeptides disclosed by the present disclosure, the raw material used is natural, safe and healthy and has a wide range of sources. The preparation method mainly includes three steps: extraction of a protein, enzymolysis and emulsification. Operation processes are simple and easy, large apparatuses are not required, and the whole technological process is green, safe and environmentally friendly. The preparation method has a low cost and can realize industrial-scale production, so that a foundation is laid for development of plant-based food ingredients.

The dual-functional bean-derived polypeptides and the emulsion thereof prepared by the present disclosure have a good antihypertensive activity (the protein concentration is 5 mg/mL polypeptide, and the ACE inhibition rate is as high as 92%), a positive effect on prevention and control of hypertension, and potential in application to pregnant women, old people and other patient populations suffered from chronic diseases. Moreover, the polypeptide and the rosemary extract also have a good antioxidant property (the DPPH free radical scavenging ability is as high as 100%), which overcome the problems of oxidation and deterioration of existing emulsions and can be widely applied in functional foods or special diet foods.

The emulsion containing bean-derived polypeptides provided by the present disclosure has good emulsibility and emulsification stability (the emulsification stability is as long as 7 days or more), which overcomes the shortcoming of poor stability of many existing protein peptide emulsions. In addition, the emulsion containing bean-derived polypeptides of the present disclosure has good oxidation stability and a long storage period, thus having a broad market application prospect.

The dual-functional polypeptide prepared by the present disclosure can simultaneously fix hydrophobic and hydrophilic functional factors or nutrients, which plays an important role in maintaining biological activities of bioactive substances and stability of nutrients and has a potential application value in foods, medicines and other fields.

### BRIEF DESCRIPTION OF FIGURES

FIG. 1 is a process flow chart showing preparation of a dual-functional polypeptide emulsion of the present disclosure.

FIG. 2A shows microscope images of crude black bean polypeptide emulsions in examples.

FIG. 2B shows microscope images of crude mung bean black bean polypeptide emulsions in examples.

FIG. 2C shows microscope images of crude red bean black bean polypeptide emulsions in examples.

FIG. 3 is a diagram showing particle size distribution of a dual-functional black bean polypeptide emulsion in Example 1.

FIG. 4 is a diagram showing particle size distribution of a dual-functional mung bean polypeptide emulsion in Example 2.

FIG. 5 is a diagram showing particle size distribution of a dual-functional red bean polypeptide emulsion in Example 3.

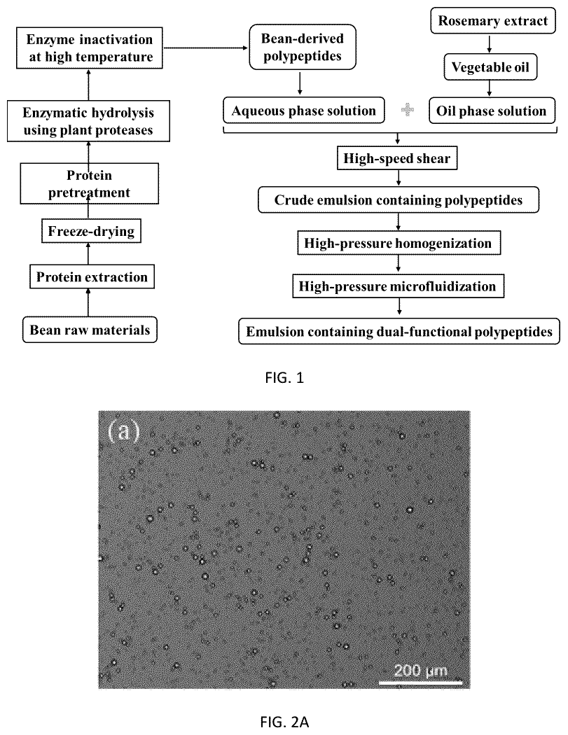

### DETAILED DESCRIPTION

Preferred examples of the present disclosure are described below. It shall be understood that the examples are intended to better explain the present disclosure, rather than to limit the present disclosure.

Biological materials: Ficin used in the examples is purchased from Sigma-Aldrich, and has a model number of 1002391235 and an enzyme activity of 1.5×10⁵ U/g; bromelain is purchased from ACROS Organics, and has a model number of 448210250 and an enzyme activity of 1×10⁵ U/g; and papain is purchased from J&K Scientific, and has a model number of 916928 and an enzyme activity of 2400 units/mg.

1. Determination of an Angiotensin Converting Enzyme (ACE) Inhibition Activity

A specific process is as follows:

Drawing of a standard curve: Hippuric acid (HA) was dissolved in a 0.05 M borate buffer (pH 8.2, containing 0.3 M NaCl) to prepare an HA standard working solution with a concentration of 3.125-100 μM. 60 μL of 1 M HCl and 120 μL of pyridine were added, then 60 μl of benzene sulfonyl chloride was added and uniformly mixed for 1 min, and cooling was performed immediately. The absorbance value was measured at a wavelength of 410 nm, and a standard curve of the absorbance value and a corresponding concentration was drawn.

Determination of an ACE activity: 12 μL of a polypeptide solution was mixed with 8 μL of ACE or a borate buffer solution (0.05 M, pH 8.3, containing 0.3 M NaCl) to obtain a group A and a group B respectively, while 12 μL of a borate buffer solution was mixed with 8 μL of ACE to obtain a group C. Each mixture was mixed with 40 μL of a substrate (prepared from 5 mM HHL and a borate buffer solution) and then incubated at 37° C. for 30 min, and then 60 μL of HCl (1 M) was added to carry out an inactivation reaction. 120 μL of pyridine and 60 μL of benzene sulfonyl chloride were sequentially added into the mixture, and the absorbance value was determined at 410 nm by a microplate reader. A calculation formula is as follows:

$$ACE inhibition activity ( \% ) = \frac{C - A}{C - B} \times 100 \% .$$

2. Evaluation of an Antioxidant Activity

(1) Determination of a DPPH free radical scavenging ability, where a method is as follows:

0.1 mM DPPH was prepared from a 95% ethanol solution, 50 μL of an enzymatic solution and 50 μL of the DPPH were added into each well of a 96-well plate to carry out a reaction at room temperature for 30 min after shaking for 10 s, and the absorbance value was measured at a wavelength of 517 nm and recorded as Asample. 50 μL of an enzymatic solution and 50 μL of 95% ethanol were used as a control group, and the absorbance value was measured under same conditions and recorded as Asample blank. 50 μL of DPPH and 50 μL of deionized water were used as a blank group, and the absorbance value was measured under same conditions and recorded as Acontrol. Each sample was subjected to 3 parallel tests to undergo a reaction at room temperature for 30 min after shaking for 30 s, and the absorbance of the sample was measured at a wavelength of 517 nm.

$$Scavenging rate ( \% ) = ( 1 - \frac{A sample - A sample blank}{A control} ) \times 100 .$$

(2) Determination of a metal ion chelating ability, where a method is as follows:

50 μL of an enzymatic solution was added to a 96-well plate, 100 μL of a 20 μM ferrous chloride solution and 100 μL of a 0.5 mM ferrozine solution were sequentially added and uniformly mixed, followed by standing at room temperature for 10 min, and then the absorbance value was measured at 562 nm. 50 μL of deionized water was used as a reference to replace the enzymatic solution, and the absorbance value was measured under same conditions and recorded as Asample blank. 50 μL of an enzymatic solution and 200 μL of deionized water were used as a blank control, and the absorbance value was measured under same conditions and recorded as Acontrol. A calculation formula is as follows:

$$Chelating rate ( \% ) = ( 1 - \frac{A sample - A sample blank}{A control} ) \times 100 .$$

3. Determination of Emulsification Properties

A method is as follows:

The emulsibility and the emulsification stability were determined by a turbidity method. 15 mL of a sample was mixed with 5 mL of an vegetable oil, followed by homogenization at 20,000 r/min at room temperature for 1 min. 50 μL of a bottom emulsion was immediately sucked and mixed with 5 mL of 0.1% SDS, and the absorption value was measured at 500 nm and recorded as A₀. After standing was performed for 60 min, the absorption value was measured by the same method and recorded as A₁, and c was set as the protein concentration of the sample. The emulsibility and the emulsification stability were calculated according to the following formulas.

Emulsibility:

$$EAI ( {m}^{2} / g ) = \frac{2 \times 2.303 \times {A}_{0}}{0.25 \times c \times 100}$$

Emulsification stability:

$$ESI ( min ) = \frac{{A}_{0} \times 60}{{A}_{0} - {A}_{1}} .$$

### Example 1

A method for preparing a dual-functional black bean polypeptide is provided. The method includes the following steps:

(1) extraction of a bean source protein isolate: subjecting black beans to pulverizing and sifting, and adding n-hexane that is 3 times of the volume of the black beans for degreasing to obtain a degreased black bean powder; dissolving the degreased black bean powder in deionized water to obtain a solution, adjusting the pH value of the solution to 9.0 with sodium hydroxide, stirring at a constant temperature of 30° C. for 1.5 h, followed by centrifugation to obtain a supernatant, and then adjusting the pH value to 4.0 with hydrochloric acid; repeating the above steps for 2 times, performing centrifugation at 10,000 rpm for 10 min to collect a protein precipitate, and washing the precipitate to neutral with deionized water; and finally, performing freeze-drying to obtain a black bean protein isolate;

(2) infrared treatment: subjecting the protein obtained above to infrared heat treatment at an infrared temperature of 120° C. for 20 min; and

(3) preparation of a polypeptide: dissolving a protein powder obtained after the infrared treatment in deionized water to prepare a solution having a substrate concentration of 12%, adjusting the pH value to 6.0, adding 2250 U/g of ficin to carry out an enzymolysis reaction in a constant-temperature reactor at 60° C. for 60 min, and after the reaction is completed, performing enzyme deactivation at a high temperature, followed by centrifugation at 8,000 rpm at 4° C. for 15 min to collect a polypeptide solution.

The ACE inhibition activity, the DPPH free radical scavenging ability and the metal ion chelating ability of the polypeptide prepared in Example 1 were determined. Determination results show that the ACE inhibition rate is 87.35%, the DPPH free radical scavenging ability is 80.36%, and the metal ion chelating ability is 95.78%. It is indicated that the polypeptide prepared in this example has a good antihypertensive activity and an antioxidant activity.

### Example 2

A method for preparing a dual-functional mung bean polypeptide is provided. The method includes the following steps:

(1) extraction of a bean source protein isolate: subjecting mung beans to pulverizing and sifting, and adding n-hexane that is 4 times of the volume of the mung beans for degreasing to obtain a degreased mung bean powder; dissolving the degreased mung bean powder in deionized water to obtain a solution, adjusting the pH value of the solution to 8.5 with sodium hydroxide, stirring at room temperature for 2 h, followed by centrifugation to obtain a supernatant, and then adjusting the pH value to 4.5 with hydrochloric acid; repeating the above steps for 2 times, performing centrifugation at 10,000 rpm for 10 min to collect a protein precipitate, and washing the precipitate to neutral with deionized water; and finally, performing freeze-drying to obtain a mung bean protein isolate;

(2) infrared treatment: subjecting the protein obtained above to infrared heat treatment at an infrared temperature of 100° C. for 25 min; and

(3) preparation of a polypeptide: dissolving a mung bean protein powder obtained after the infrared treatment in deionized water to prepare a solution having a substrate concentration of 10%, adjusting the pH value to 7.0, adding 2500 U/g of bromelain to carry out an enzymolysis reaction in a constant-temperature reactor at 55° C. for 3 h, and after the reaction is completed, performing enzyme deactivation at a high temperature, followed by centrifugation at 8,000 rpm at 4° C. for 15 min to collect a polypeptide solution.

The ACE inhibition activity, the DPPH free radical scavenging ability and the metal ion chelating ability of the polypeptide prepared in Example 2 were determined. Determination results show that the ACE inhibition rate is 91.87%, the DPPH free radical scavenging ability is 84.63%, and the metal ion chelating ability is 98.74%. It is indicated that the polypeptide prepared in this example has a good antihypertensive activity and an antioxidant activity.

### Example 3

A method for preparing a dual-functional red bean polypeptide is provided. The method includes the following steps:

(1) extraction of a bean source protein isolate: subjecting red beans to pulverizing and sifting, and adding n-hexane that is 4 times of the volume of the red beans for degreasing to obtain a degreased red bean powder; dissolving the degreased red bean powder in deionized water to obtain a solution, adjusting the pH value of the solution to 9.0 with sodium hydroxide, stirring at room temperature for 2 h, followed by centrifugation to obtain a supernatant, and then adjusting the pH value to 4.3 with hydrochloric acid; repeating the above steps for 2 times, performing centrifugation at 10,000 rpm for 10 min to collect a protein precipitate, and washing the precipitate to neutral with deionized water; and finally, performing freeze-drying to obtain a red bean protein isolate;

(2) infrared treatment: subjecting the protein obtained above to infrared heat treatment at an infrared temperature of 100° C. for 20 min; and

(3) preparation of a polypeptide: dissolving a red bean protein powder obtained after the infrared treatment in deionized water to prepare a solution having a substrate concentration of 10%, adjusting the pH value to 6.5, adding 4000 U/g of papain to carry out an enzymolysis reaction in a constant-temperature reactor at 55° C. for 4 h, and after the reaction is completed, performing enzyme deactivation at a high temperature, followed by centrifugation at 8,000 rpm at 4° C. for 15 min to collect a polypeptide solution.

The ACE inhibition activity, the DPPH free radical scavenging ability and the metal ion chelating ability of the polypeptide prepared in Example 3 were determined. Determination results show that the ACE inhibition rate is 92.55%, the DPPH free radical scavenging ability is 81.39%, and the metal ion chelating ability is 94.31%. It is indicated that the polypeptide prepared in this example has a good antihypertensive activity and an antioxidant activity.

### Example 4

A method for preparing bean-derived polypeptides emulsion is provided. The method includes the following steps:

preparation of a polypeptide emulsion by using the bean-derived polypeptides prepared in Examples 1-3 as a raw material separately, including the following steps:

(1) pre-preparation of a crude polypeptide emulsion: adjusting a polypeptide solution to a protein concentration of 5 mg/mL to serve as an aqueous phase solution, dissolving 0.15 g/kg of a rosemary extract in a sunflower seed oil to obtain an oil phase, mixing the aqueous phase with the oil phase at a mass ratio of 4:1, and performing high-speed shear dispersion at a rotation speed of 20,000 rpm for 5 min to obtain a crude emulsion; and

(2) preparation of a polypeptide emulsion: subjecting the crude emulsion obtained above to pretreatment immediately by a high-pressure homogenizer at a pressure of 150/30 bar, rapidly cooling the emulsion to 5° C. in an ice water bath immediately after homogenization is completed, and then performing ultrahigh-pressure micro-fluidization treatment at a pressure of 1,000 bar, followed by circulation for 3 times to obtain a corresponding polypeptide nanoemulsion.

1. Results of a polypeptide emulsion prepared by using the black bean polypeptide prepared in Example 1 are as follows:

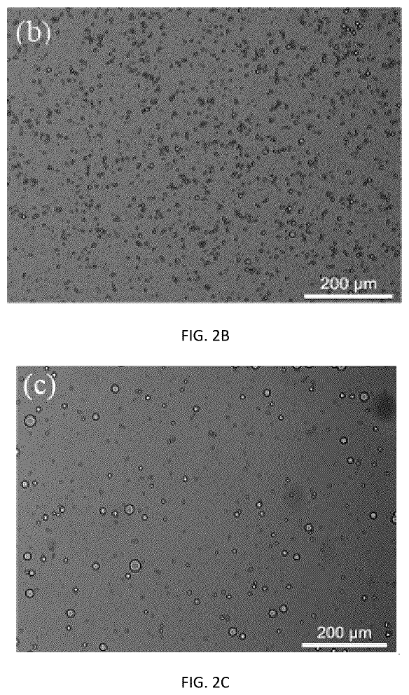

Emulsification properties of the crude polypeptide emulsion obtained in step (1) were determined, and microscopic morphology of the crude polypeptide emulsion was observed. Determination results show that the emulsion has an emulsification index of 24.17 m²/g, an emulsification stability of 983.94 min, uniform distribution and a particle size maintained at about 10 μm (FIG. 2a).

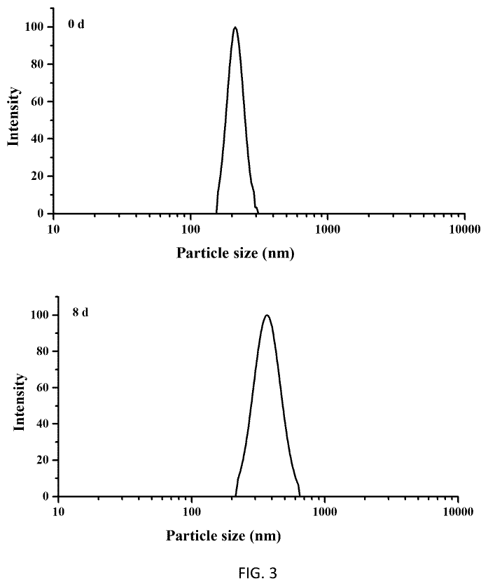

The particle size and stability of the polypeptide nanoemulsion obtained in step (2) were determined. Determination results show that the polypeptide nanoemulsion has an average particle size of 311.47 nm and an average particle size of 406.09 nm after storage for 8 d, is maintained relatively stable, and has a secondary oxidation product inhibition rate as high as 88.61% and good oxidation stability (FIG. 3).

2. Results of a polypeptide emulsion prepared by using the mung bean polypeptide prepared in Example 2 are as follows:

Emulsification properties of the crude polypeptide emulsion obtained in step (1) were determined, and microscopic morphology of the crude polypeptide emulsion was observed. Determination results show that the emulsion has an emulsification index of 52.45 m²/g, an emulsification stability of 4686.63 min, uniform distribution and a particle size maintained at about 5 μm (FIG. 2b).

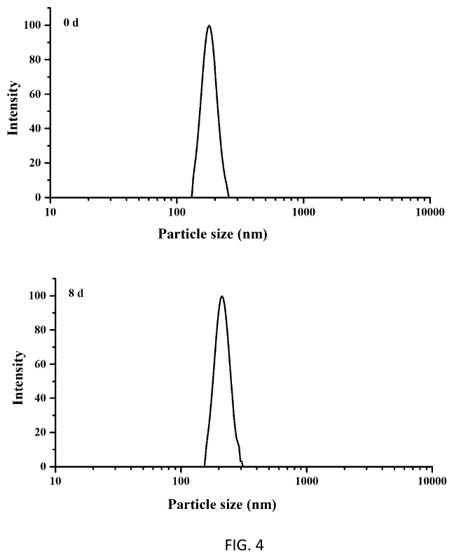

The particle size and stability of the polypeptide nanoemulsion obtained in step (2) were determined. Determination results show that the polypeptide nanoemulsion has an average particle size of 261.17 nm and an average particle size of 311.47 nm after storage for 8 d, is maintained relatively stable, and has a secondary oxidation product inhibition rate as high as 90.33% and good oxidation stability (FIG. 4).

3. Results of a polypeptide emulsion prepared by using the red bean polypeptide prepared in Example 3 are as follows:

Emulsification properties of the crude polypeptide emulsion obtained in step (1) were determined, and microscopic morphology of the crude polypeptide emulsion was observed. Determination results show that the emulsion has an emulsification index of 21.85 m²/g, an emulsification stability of 897.63 min, uniform distribution and a particle size maintained at about 15 μm (FIG. 2c).

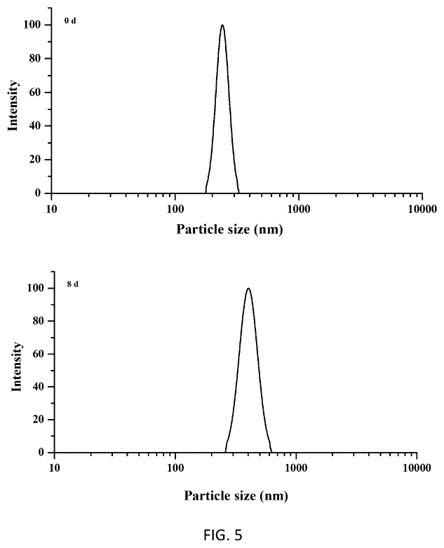

The particle size and stability of the polypeptide nanoemulsion obtained in step (2) were determined. Determination results show that the polypeptide nanoemulsion has an average particle size of 370.13 nm and an average particle size of 495.60 nm after storage for 8 d, is maintained relatively stable, and has a secondary oxidation product inhibition rate as high as 86.27% and good oxidation stability (FIG. 5).

### Example 5

A method for preparing bean-derived polypeptides microcapsule powder is provided. The method includes the following steps:

subjecting the emulsion containing bean-derived polypeptides obtained in Example 4 to spray-drying at an inlet temperature of 140° C. and an outlet temperature of 75° C., and after the drying is completed, obtaining a polypeptide microcapsule powder.

The polypeptide microcapsule powder prepared in this example is milky white and has a uniform particle size and good oxidation stability.

### Comparative Example 1

A black bean polypeptide is prepared with reference to the method in Example 1. The difference is that the sequence of the infrared treatment is adjusted. The black bean powder is subjected to the infrared treatment first and then subjected to the alkali-solution and acid-isolation treatment. Other conditions are the same as those in Example 1.

The black bean polypeptide prepared has an ACE inhibition rate of 4.29%, a DPPH free radical scavenging ability of 62.31% and a metal ion chelating ability of 70.08%. Furthermore, an emulsion prepared from the black bean polypeptide is unstable, and has a secondary oxidation product inhibition rate of only 12.56% and extremely poor oxidation stability.

### Comparative Example 2

A black bean polypeptide is prepared with reference to the method in Example 2. The difference is that conditions of the infrared treatment are adjusted (see Table 1). Other conditions are the same as those in Example 1. Results are shown in Table 1.

TABLE 1
Effects of different infrared conditions
on functions of a polypeptide

| | | | DPPH free | |
|---|---|---|---|---|
| Infrared | Infrared | ACE | radical | Metal ion |
| temperature | time | inhibition | scavenging | chelating |
| (° C.) | (min) | rate (%) | ability (%) | ability (%) |
| 50 | 20 | — | 10.76 | 10.49 |
| 60 | 20 | — | 11.25 | 10.36 |
| 150 | 20 | 49.30 | 69.04 | 71.03 |
| 160 | 20 | 13.51 | 65.62 | 60.21 |
| 110 | 5 | 7.22 | 25.03 | 27.81 |
| 110 | 120 | 23.51 | 42.31 | 48.27 |

By summarizing and analyzing the data in Table 1, it can be found that when the infrared treatment is performed at a too high temperature for too long time, the structure of a bean source protein is seriously damaged, and the ACE inhibition rate and the antioxidant activity are both limited; and when the infrared treatment is performed at a too low temperature for too short time, a bean source protein fragment cannot be effectively subjected to enzymolysis with a protease, and obtained peptide segments having an ACE inhibition activity and an antioxidant activity are few. Therefore, preferably, the infrared treatment is performed at an infrared temperature of 70-140° C. for 10-60 min.

### Comparative Example 3

A black bean polypeptide is prepared with reference to the method in Example 1. The difference is that the infrared treatment is omitted (namely, the step (2) is omitted). Other conditions are the same as those in Example 1.

### Comparative Example 4

A black bean polypeptide is prepared with reference to the method in Example 1. The difference is that a protease is not added (namely, the step (3) is omitted). Other conditions are the same as those in Example 1.

### Comparative Example 5

A black bean polypeptide emulsion is prepared with reference to the method in Example 4. The difference is that the ultrahigh-pressure micro-fluidization treatment is not performed. Other conditions are the same as those in Example 4.

TABLE 2
Comparison of effects of different treatments on an emulsion containing
black bean-derived polypeptides.

| | | DPPH | | | | | Oxidation |
|---|---|---|---|---|---|---|---|
| | | free | Metal | | | | product |
| | ACE | radical | ion | | | Particle | inhibition |
| | inhibition | scavenging | chelating | | Emulsification | size of a | rate of a |
| | rate | ability | ability | Emulsibility | stability | nanoemulsion | nanoemulsion |
| Sample | (%) | (%) | (%) | (m²/g) | (min) | (nm) | (%) |
| Example 1 | 87.35 | 80.36 | 95.78 | 24.17 | 983.94 | 311.47 | 88.61 |
| Comparative | — | 10.21 | 13.05 | 16.73 | 164.22 | 400.91 | 35.42 |
| Example 3 | | | | | | | |
| Comparative | — | 4.76 | 6.73 | 13.37 | 388.67 | 451.83 | 19.79 |
| Example 4 | | | | | | | |
| Comparative | 87.35 | 80.36 | 95.78 | 24.17 | 983.94 | 431.15 | 69.07 |
| Example 5 | | | | | | | |

By summarizing and analyzing the data in the above table, it can be found that a bean source protein without the infrared treatment has a spherical folded structure, a protease cannot be effectively hydrolyzed, peptide segments prepared have larger molecular weights, and obtained peptide segments having an ACE inhibition activity and an antioxidant activity are obviously few, so that the particle size and oxidation stability of an emulsion are affected. Without an enzymolysis effect of a protease, a bean source protein cannot produce active peptide segments effectively. Without the ultrahigh-pressure microfluidization treatment during preparation of a nanoemulsion, the nanoemulsion prepared has a larger particle size and poor stability, so that distribution of a peptide and a rosemary extract in an oil or aqueous phase is affected, and the oxidation stability is further affected.

### Comparative Example 6

Mung bean polypeptide emulsions are prepared with reference to the method in Example 4. The differences are that different concentrations of the mung bean polypeptide prepared in Example 2 are separately used as an aqueous phase solution, and different concentrations of a rosemary extract are separately dissolved in a sunflower seed oil to obtain an oil phase. Other conditions are the same as those in Example 4, and an emulsion containing mung bean-derived polypeptides are prepared.

The particle size, potential and centrifugal stability of the emulsion containing mung bean-derived polypeptides were determined. The particle size and the potential were determined by a laser particle size analyzer. The centrifugal stability of the emulsions was characterized by determining the emulsification index. A higher emulsification index indicates stronger stability. Specific steps are as follows: placing an emulsion in a centrifuge tube, performing centrifugation at 13,000 g for 2 min, and measuring and recording the height of the emulsion before and after the centrifugation. Results are shown in Table 3.

TABLE 3
Properties of polypeptide emulsion containing different
concentrations of mung bean-derived polypeptides and
different concentrations of rosemary extract.

| Concentration | | | | |
|---|---|---|---|---|
| of a | Concentration | Particle size | | Emulsifi- |
| polypeptide | of a rosemary | of an | Potential | cation |
| (mg/mL) | extract (g/kg) | emulsion | (mV) | index (%) |
| 5 | 0 | 566.02 | −14.3 | 53.28 |
| | 0.05 | 352.10 | −20.3 | 55.09 |
| | 0.10 | 339.54 | −28.0 | 70.20 |
| | 0.15 | 311.47 | −34.5 | 82.63 |
| | 0.20 | 322.59 | −35.2 | 83.55 |
| 0.5 | 0.15 | 550.72 | −12.4 | 18.39 |
| 1 | | 423.64 | −21.8 | 67.55 |
| 5 | | 311.47 | −34.5 | 82.63 |
| 10 | | 309.28 | −33.9 | 81.77 |
| 15 | | 320.04 | −34.1 | 81.34 |

Results in the above table show that a synergistic interaction of the polypeptide and the rosemary extract can not only improve the oxidation stability of an emulsion, but also is conducive to improving the physical stability of the emulsion. This is mainly because the peptide and the rosemary extract are combined in the form of a hydrogen bond to form a stable amphiphilic complex, which is easier to maintain stability at an oil-water interface. Therefore, the emulsification index of the emulsion is improved, the emulsion has a smaller particle size and a relatively higher potential, and the emulsion is better dispersed and has good emulsification stability.

Although the present disclosure has been disclosed above as preferred examples, the examples are not intended to limit the present disclosure, and various changes and modifications can be made by any person familiar with the technology without departing from the spirit and scope of the present disclosure. Therefore, the protection scope of the present disclosure shall be defined by the claims.

What is claimed is:

1. A method for preparing emulsion containing bean-derived polypeptides, wherein the emulsion is obtained by homogenization and mixing of dual-functional bean-derived polypeptides and an oil phase.

2. The method according to claim 1, wherein a method for preparing dual-functional bean-derived polypeptides comprises the following steps:
(1) extraction of a bean source protein isolate: subjecting a bean raw material to pulverizing and sifting, adding n-hexane for degreasing to obtain a degreased bean powder, then extracting a protein from the degreased bean powder by an alkali-solution and acid-isolation method, and performing freeze-drying to obtain a bean source protein isolate;
(2) infrared treatment: subjecting the bean source protein obtained in step (1) to infrared heat treatment; and
(3) preparation of a polypeptide: dissolving an infrared protein powder obtained in step (2) in deionized water to prepare a solution having a substrate concentration of 5-20%, adjusting the pH value, adding a protease to carry out an enzymolysis reaction, and after the reaction is completed, performing enzyme deactivation at a high temperature, followed by centrifugation to collect a polypeptide solution.

3. The method according to claim 2, wherein the bean raw material comprises one or more of protein-rich soybeans, black beans, peas, red beans, or mung beans.

4. The method according to claim 2, wherein in step (1), a ratio of the material to the n-hexane during the degreasing is 1:3 to 1:10 (g/mL); and the alkali-solution and acid-isolation method specifically comprises: dissolving the degreased bean powder in deionized water to obtain a solution, adjusting the pH value of the solution to 7.2-10.5 with sodium hydroxide, stirring at a constant temperature of 25-50° C. for 0.5-4.5 hours, followed by centrifugation to obtain a supernatant, then adjusting the pH value to 2.0-5.5 with hydrochloric acid, repeating the above steps for 2-3 times to obtain a precipitate, and washing the precipitate to neutral with deionized water.

5. The method according to claim 2, wherein in step (3), the protease comprises one or more of ficin, bromelain and papain, the protease is added in an amount of 500-5,000 U/g protein powder, and the enzymolysis reaction is carried out at a temperature of 35-65° C. and a pH value of 4.5-7.5 for 0.5-8 h.

6. The emulsion containing bean-derived polypeptides prepared by the method according to claim 1.

7. A composition containing the emulsion according to claim 6.

8. Application of the emulsion according to claim 6 in preparation of foods, health care products and cosmetics.

9. A dual-functional bean-derived polypeptides microcapsule powder, wherein a preparation method therefor comprises subjecting the polypeptide emulsion according to claim 6 to spray-drying treatment to obtain the dual-functional bean-derived polypeptides microcapsule powder.
